(19) United States
(12) Patent Application Publication
(10) Pub. No.: US 2024/0156854 A1
(43) Pub. Date: May 16, 2024

(54) METHYLCOBALAMIN OPHTHALMIC PREPARATION AND USE THEREOF

(71) Applicant: EYE INSTITUTE OF SHANDONG FIRST MEDICAL UNIVERSITY, Qingdao (CN)

(72) Inventors: Weiyun Shi, Qingdao (CN); Bi Ning Zhang, Qingdao (CN); Shijiu Chen, Qingdao (CN); Qingjun Zhou, Qingdao (CN); Hongwei Wang, Qingdao (CN)

(21) Appl. No.: 18/295,247

(22) Filed: Apr. 3, 2023

(30) Foreign Application Priority Data
Nov. 14, 2022 (CN) 202211423845.2

(51) Int. Cl. A61K 31/714; A61K 9/00; A61K 31/353; A61K 47/10; A61K 47/38; A61K 47/42; A61P 27/02

(52) U.S. Cl. CPC A61K 31/714; A61K 9/0048; A61K 31/353; A61K 47/10; A61K 47/38; A61K 47/42; A61P 27/02

(57) ABSTRACT

The present disclosure provides a methylcobalamin ophthalmic preparation and a preparation method and use thereof, belonging to the technical field of eye drops. The methylcobalamin ophthalmic preparation includes a methylcobalamin technical (TC) and proanthocyanidin, where the methylcobalamin TC and the proanthocyanidin have a mass ratio of (0.02-0.1):(0-0.5); and a dosage form of the methylcobalamin ophthalmic preparation is one or more of a drop, a hydrogel, and a liposome. In the present disclosure, the methylcobalamin is used as an active ingredient, and the proanthocyanidin is used as a synergist of the methylcobalamin; the methylcobalamin ophthalmic preparation can promote corneal nerve regeneration, accelerate corneal epithelial healing, and effectively inhibit corneal neovascularization to improve corneal perception. The methylcobalamin ophthalmic preparation solves complications caused by long-term systemic use of the methylcobalamin, makes up for a gap of low-cost drugs in the field of corneal nerve regeneration, and increases a selection range of non-invasive drugs.

### CROSS REFERENCE TO RELATED APPLICATION

This patent application claims the benefit and priority of Chinese Patent Application No. 202211423845.2, filed with the China National Intellectual Property Administration on Nov. 14, 2022, the disclosure of which is incorporated by reference herein in its entirety as part of the present application.

### TECHNICAL FIELD

The present disclosure relates to the technical field of ophthalmic preparations, in particular to a methylcobalamin (mecobalamin) ophthalmic preparation and use thereof.

### BACKGROUND

Neurotrophic keratitis is a degenerative disease concerning corneal epithelial disorder during peripheral paralysis of the trigeminal nerve, which could lead to blindness. Trigeminal paralysis can be caused by factors such as intracranial surgery, injection of drugs, tumor, skull fracture, and infection. The trigeminal paralysis can also occur in the central nervous system, accompanied by neurotrophic keratitis with related symptoms of facial nerve palsy. Clinically, neurotrophic keratitis can be divided into three stages according to the severity: abnormal tear film and epithelial changes (stage I), persistent epithelial defects (stage II), and corneal ulceration (stage III). The principle of treatment is to block the progress of corneal damage. For mild stage I, ocular lubricants (such as artificial tears without preservatives) are clinically administered instead of using ophthalmic preparations. For moderate stage II, corneal bandage lens or single-layer amniotic membrane transplantation or autologous serum eye drops are recommended. For severe stage III cases, surgical methods such as palpebral fissure suture, conjunctival flap covering, and multilayer amniotic membrane transplantation are conducted to restore the integrity of the ocular surface. Currently, traditional treatments mainly focus on symptomatic treatments, which fail to solve the fundamental problem of corneal nerve injury. When the traditional treatments failed to restore the function of the cornea, corneal transplantation is the last salvage option. However, since the neurotrophic problem has not been fundamentally resolved, it is difficult for the graft to survive after keratoplasty.

Methylcobalamin is a derivative of vitamin B12. Compared with inactive vitamin B12, the methylcobalamin is more likely to enter nerve organelles. Methylcobalamin can participate in the methyl conversion of substances and the metabolism of nucleic acids, proteins, and lipids, and promote the synthesis of nucleic acids and proteins in nerve cells as well as nerve myelin, therefore to repair the damaged peripheral nerves. In view of its desirable neuroprotective effect, methylcobalamin has been clinically used in the treatment of ophthalmic diseases such as glaucoma, diabetic retinopathy, central serous chorioretinopathy, optic nerve injury, and external ophthalmoplegia.

At present, methylcobalamin is commonly applied by oral intake or injection to treat ophthalmic diseases. Neither the oral preparations nor injection shows sufficient effects in improving neural regeneration. Moreover, long-term systemic application of methylcobalamin is prone to complications. For example, long-term oral administration of methylcobalamin may lead to increased nerve sensitivity, resulting in frequent headaches in patients, and can also cause local paresthesias, frequent fever, and even sweating symptoms. Intramuscular injection of methylcobalamin may cause severe pain at the injection site, and long-term injection may increase the possibility of local thrombus, which can affect normal blood circulation, and eventually lead to rough, dry, and even hard skin on the local skin surface.

### SUMMARY

In view of this, an objective of the present disclosure is to provide a methylcobalamin ophthalmic preparation. In the present disclosure, the methylcobalamin ophthalmic preparation has a desirable curative effect on neurotrophic keratitis by local application to avoid complications caused by long-term systemic application.

To achieve the above objective of the present disclosure, the present disclosure provides the following technical solutions.

The present disclosure provides a methylcobalamin ophthalmic preparation, including a methylcobalamin technical (TC) and proanthocyanidin, where the methylcobalamin TC and the proanthocyanidin have a mass ratio of (0.02-0.1):(0-0.5); and a dosage form of the methylcobalamin ophthalmic preparation is one or more of a drop, a hydrogel, and a liposome.

Preferably, when the dosage form is the drop, the methylcobalamin ophthalmic preparation includes the following components by percentage:
the methylcobalamin TC 0.02% to 0.1%;
the proanthocyanidin 0.1% to 0.5%;
a buffer 0.1% to 1.0%;
an osmotic pressure regulator 0.1% to 5.0%;
a pH regulator 0.001% to 0.004%; and
water as a balance.

Preferably, the buffer is one or more selected from the group consisting of sodium hyaluronate, disodium hydrogen phosphate, sodium dihydrogen phosphate, potassium dihydrogen phosphate, and sodium citrate.

Preferably, the osmotic pressure regulator is one or more selected from the group consisting of propylene glycol, glycerin, and polyethylene glycol.

Preferably, the pH regulator is one or more selected from the group consisting of citric acid, sodium citrate, boric acid, disodium hydrogen phosphate, sodium dihydrogen phosphate, acetic acid, sodium acetate, sodium hydroxide, and hydrochloric acid.

Preferably, when the dosage form is the hydrogel, the methylcobalamin ophthalmic preparation includes the following components by percentage:
the methylcobalamin TC 0.02% to 0.1%;
the proanthocyanidin 0.1% to 0.5%;
gelatin 4.5% to 5.5%;
carboxymethyl cellulose 0.45% to 0.55%;
an N-hydroxysulfosuccinimide sodium salt 0.17% to 0.18%; and
water for injection as a balance.

Preferably, when the dosage form is the liposome, the methylcobalamin ophthalmic preparation includes the following components by percentage:
the methylcobalamin TC 0.02% to 0.1%;
the proanthocyanidin 0.1% to 0.5%;
lecithin 0.08% to 0.15%;
cholesterol 0.02% to 0.05%; and
water as a balance.

Preferably, the methylcobalamin ophthalmic preparation has a pH value of 6 to 8.

Preferably, when the dosage form is the drop, the methylcobalamin ophthalmic preparation has an osmolality of 260 mOsm/kg to 320 mOsm/kg.

The present disclosure further provides a method for treating neurotrophic keratitis by using the above-described methylcobalamin ophthalmic preparation.

The present disclosure provides a methylcobalamin ophthalmic preparation, including a methylcobalamin technical (TC) and proanthocyanidin, where the methylcobalamin TC and the proanthocyanidin have a mass ratio of (0.02-0.1):(0-0.5); and a dosage form of the methylcobalamin ophthalmic preparation is one or more of a drop, a hydrogel, and a liposome. In the present disclosure, methylcobalamin is used as an active ingredient. Methylcobalamin is one of two activated coenzyme forms of vitamin B12. Methylcobalamin can enhance the methylation of neurons, accelerate the growth of nerve cells, and reduce the level of homocysteine, showing a desirable neuroprotective effect. The proanthocyanidin is used as a synergist of the methylcobalamin. Proanthocyanidins contain a variety of polyphenolic compounds, and are widely found in the flowers, bark, peels, and seeds of many plants. At present, proanthocyanidins have been proved to have various beneficial activities, and its own specific chemical structure brings strong anti-oxidation, anti-bacterial, anti-inflammatory and anti-diabetic properties and other health-promoting activities. Proanthocyanidins can protect damaged retinal ganglion cells and inhibit retinal neovascularization. The proanthocyanidin and the methylcobalamin have a synergistic effect, not only can enhance the repair of corneal nerves, but also can further inhibit corneal neovascularization. The methylcobalamin ophthalmic preparation can promote corneal nerve regeneration, accelerate corneal epithelial healing, and effectively inhibit corneal neovascularization to improve corneal perception. The methylcobalamin ophthalmic preparation solves complications caused by long-term systemic use of the methylcobalamin. The ophthalmic preparation provides a new option for the treatment of neurotrophic keratitis, with a low cost.

### BRIEF DESCRIPTION OF THE DRAWINGS

FIG. 1 shows an electron micrograph of a methylcobalamin liposome;

FIG. 2 shows a general photo of the eyes of experimental mice; and

FIGS. 3A-D show imaging results of a confocal laser microscope of the cornea.

### DETAILED DESCRIPTION OF THE EMBODIMENTS

The present disclosure provides a methylcobalamin ophthalmic preparation, including a methylcobalamin technical (TC) and proanthocyanidin, where the methylcobalamin TC and the proanthocyanidin have a mass ratio of (0.02-0.1):(0-0.5), preferably (0.02-0.1):(0.1-0.5), more preferably (0.05-0.08):(0.2-0.4); and a dosage form of the methylcobalamin ophthalmic preparation is one or more of a drop, a hydrogel, and a liposome.

In the present disclosure, the methylcobalamin ophthalmic preparation has a pH value of preferably 6 to 8, more preferably 6.5 to 7.5.

In the present disclosure, when the dosage form is the drop, the methylcobalamin ophthalmic preparation includes the following components by percentage:
the methylcobalamin TC 0.02% to 0.1%;
the proanthocyanidin 0.1% to 0.5%;
a buffer 0.1% to 1.0%;
an osmotic pressure regulator 0.1% to 5.0%;
a pH regulator 0.001% to 0.004%; and
water for injection as a balance.

In the present disclosure, when the dosage form is the drop, the methylcobalamin ophthalmic preparation is called a methylcobalamin ophthalmic drop.

In the present disclosure, the methylcobalamin ophthalmic drop includes 0.02% to 0.1%, preferably 0.05% to 0.08% of the methylcobalamin TC by mass percent. The methylcobalamin TC has a purity of preferably 0.05%. In the present disclosure, methylcobalamin is used as an active ingredient. Methylcobalamin is one of two activated coenzyme forms of vitamin B12. Methylcobalamin can enhance the methylation of neurons, accelerate the growth of nerve cells, and reduce the level of homocysteine, showing a desirable neuroprotective effect.

In the present disclosure, the methylcobalamin ophthalmic drop includes 0.1% to 0.5%, preferably 0.3% to 0.4% of the proanthocyanidin by mass percent. The proanthocyanidin is a common plant extract containing a variety of polyphenolic compounds and is widely present in flowers, bark, peels, and seeds of various plants. At present, proanthocyanidins have been proved to have various beneficial activities, and its own specific chemical structure brings strong anti-oxidation, anti-bacterial, anti-inflammatory and anti-diabetic properties, and other health-promoting activities. Proanthocyanidins can protect damaged retinal ganglion cells and inhibit retinal neovascularization.

In the present disclosure, the methylcobalamin ophthalmic drop includes 0.1% to 1.0%, preferably 0.4% to 0.6% of the buffer by mass percent. The buffer is preferably one or more selected from the group consisting of sodium hyaluronate, disodium hydrogen phosphate, sodium dihydrogen phosphate, potassium dihydrogen phosphate, and sodium citrate. The buffer can reduce irritation, increase stability, and enhance the therapeutic effect.

In the present disclosure, the methylcobalamin ophthalmic drop includes 0.1% to 5.0%, preferably 1.0% to 3.0% of the osmotic pressure regulator by mass percent. The osmotic pressure regulator is preferably one or more selected from the group consisting of ethanol, propylene glycol, glycerin, and polyethylene glycol. The polyethylene glycol has a molecular weight of preferably 200 to 400. The methylcobalamin ophthalmic drop has an osmolality of preferably 260 mOsm/kg to 320 mOsm/kg, more preferably 280 mOsm/kg to 300 mOsm/kg.

In the present disclosure, the methylcobalamin ophthalmic drop includes 0.001% to 0.004% of the pH regulator by mass percent. The pH regulator is preferably one or more selected from the group consisting of citric acid, sodium citrate, boric acid, disodium hydrogen phosphate, sodium dihydrogen phosphate, acetic acid, sodium acetate, sodium hydroxide, and hydrochloric acid.

In the present disclosure, the methylcobalamin ophthalmic drop includes water for injection as a balance by mass percent.

In the present disclosure, a preparation method of the methylcobalamin ophthalmic drop includes preferably the following steps:
dissolving the proanthocyanidin, the buffer, and the osmotic pressure regulator in part of the water for injection, and then adding the methylcobalamin, the pH regulator and the rest of the water for injection to obtain the methylcobalamin ophthalmic drop.

In the present disclosure, the part of the water for injection and the rest of the water for injection have a volume ratio of preferably 3-7:7-3, more preferably 1:1.

In the present disclosure, when the dosage form is the hydrogel, the methylcobalamin ophthalmic preparation includes the following components by percentage:
the methylcobalamin TC 0.02% to 0.1%;
the proanthocyanidin 0.1% to 0.5%;
gelatin 4.5% to 5.5%;
carboxymethyl cellulose 0.45% to 0.55%;
an N-hydroxysulfosuccinimide sodium salt 0.17% to 0.18%; and
water as a balance.

In the present disclosure, when the dosage form is the hydrogel, the methylcobalamin ophthalmic preparation is called a methylcobalamin ophthalmic hydrogel preparation.

In the present disclosure, the methylcobalamin ophthalmic hydrogel preparation includes 0.02% to 0.1%, preferably 0.05% to 0.08% of the methylcobalamin TC by mass percent. The methylcobalamin TC has a purity of preferably 0.05%.

In the present disclosure, the methylcobalamin ophthalmic hydrogel preparation includes 0.1% to 0.5%, preferably 0.3% to 0.4% of the proanthocyanidin by mass percent.

In the present disclosure, the methylcobalamin ophthalmic hydrogel preparation includes 4.5% to 5.5%, preferably 5% of the gelatin by mass percent.

In the present disclosure, the methylcobalamin ophthalmic hydrogel preparation includes 0.45% to 0.55%, preferably 0.5% of the carboxymethyl cellulose by mass percent.

In the present disclosure, the methylcobalamin ophthalmic hydrogel preparation includes 0.17% to 0.18%, preferably 0.175% of the N-hydroxysulfosuccinimide sodium salt by mass percent.

In the present disclosure, the methylcobalamin ophthalmic hydrogel preparation includes water for injection as a balance by mass percent.

In the present disclosure, a preparation method of the methylcobalamin ophthalmic hydrogel preparation includes preferably the following steps:
conducting ultrasonic mixing on the methylcobalamin TC, the proanthocyanidin, the gelatin, and water to obtain a mixed solution; and
mixing the mixed solution with the carboxymethyl cellulose and the N-hydroxysulfosuccinimide sodium salt, and conducting cross-linking to obtain the methylcobalamin ophthalmic hydrogel preparation.

In the present disclosure, the ultrasonic mixing is conducted at a power of preferably 200 W to 400 W, more preferably 300 W for preferably 20 min to 30 min, more preferably 25 min.

In the present disclosure, the ultrasonic mixing is preferably at 30° C. to 50° C., more preferably at 40° C.

In the present disclosure, the crosslinking is conducted at preferably a room temperature for preferably 1 h to 2 h, more preferably 1.5 h.

In the present disclosure, when the dosage form is the liposome, the methylcobalamin ophthalmic preparation includes the following components by percentage:
the methylcobalamin TC 0.02% to 0.1%;
the proanthocyanidin 0.1% to 0.5%;
lecithin 0.08% to 0.15%;
cholesterol 0.02% to 0.05%; and
water as a balance.

In the present disclosure, when the dosage form is the liposome, the methylcobalamin ophthalmic preparation is called a methylcobalamin ophthalmic liposome preparation.

In the present disclosure, the methylcobalamin ophthalmic liposome preparation includes 0.02% to 0.1%, preferably 0.05% to 0.08% of the methylcobalamin TC by mass percent. The methylcobalamin TC has a purity of preferably 0.05%.

In the present disclosure, the methylcobalamin ophthalmic liposome preparation includes 0.1% to 0.5%, preferably 0.3% to 0.4% of the proanthocyanidin by mass percent.

In the present disclosure, the methylcobalamin ophthalmic liposome preparation includes 0.08% to 0.15%, preferably 0.1% to 0.12% of the lecithin by mass percent.

In the present disclosure, the methylcobalamin ophthalmic liposome preparation includes 0.02% to 0.05%, preferably 0.03% to 0.04% of the cholesterol by mass percent.

In the present disclosure, the methylcobalamin ophthalmic liposome preparation includes water as a balance by mass percent. The water is preferably pure water.

In the present disclosure, a preparation method of the methylcobalamin ophthalmic liposome preparation includes preferably the following steps:
conducting ultrasonic mixing on the methylcobalamin TC, the proanthocyanidin, the lecithin, the cholesterol, and an organic solvent, and removing the organic solvent to obtain a liposome film; and
conducting ultrasonic mixing on the liposome film with the buffer for solid-liquid separation, mixing an obtained solid with water, and conducting microfiltration to obtain the methylcobalamin ophthalmic preparation.

In the present disclosure, the organic solvent is preferably a mixture of an alcohol solvent and chloroform, and the alcohol solvent is preferably methanol and/or ethanol; the alcohol solvent and the chloroform have a volume ratio of preferably 1:3.

In the present disclosure, the ultrasonic mixing is conducted at a power of preferably 200 W for preferably 10 min.

In the present disclosure, the organic solvent is removed preferably by vacuum distillation. The vacuum distillation is conducted preferably under stirring at preferably 100 rpm. The organic solvent is removed at preferably 30° C.

In the present disclosure, the buffer is preferably a PBS solution. The ultrasonic mixing is conducted at a power of preferably 200 W for preferably 15 min to 20 min.

In the present disclosure, the solid-liquid separation is conducted preferably by centrifugation.

In the present disclosure, a microfiltration membrane used in the microfiltration has a pore size of preferably 0.22 μm.

The present disclosure further provides a method for treating neurotrophic keratitis by using the methylcobalamin ophthalmic preparation.

The methylcobalamin ophthalmic preparation and the use thereof provided by the present disclosure will be described in detail below in conjunction with examples, but they should not be construed as limiting the protection scope of the present disclosure.

### Example 1

A methylcobalamin ophthalmic drop included the following components:
a methylcobalamin TC 0.05%;
proanthocyanidin 0.3%;
a buffer 0.1%;
an osmotic pressure regulator 0.5%;
a pH regulator 0.002%; and
water for injection as a balance; where

the methylcobalamin ophthalmic preparation had a pH value of 6.5.

A preparation method of the methylcobalamin ophthalmic drop included the following steps:
(1) the methylcobalamin TC, the proanthocyanidin, sodium hyaluronate, and polyethylene glycol 400 were dissolved in 0.5 ml of water for injection; and
(2) acetic acid and 0.5 ml of water for injection were added, stirred well, and adjusted to a pH value of 6.0 to 8.0; a drug solution was filtered and sterilized to pass the inspection, 0.3 mL to 0.5 mL of the drug solution was filled into a 1 mL single-dose and light-proof packaging plastic container in a 100-grade environment, and sealed to obtain a finished methylcobalamin ophthalmic preparation.

### Example 2

A methylcobalamin ophthalmic hydrogel preparation included the following components:
a methylcobalamin TC 0.05%;
proanthocyanidin 0.3%;
gelatin 5%;
carboxymethyl cellulose 0.45%;
an N-hydroxysulfosuccinimide sodium salt 0.18%; and
water for injection 1 ml.

A preparation method of the methylcobalamin ophthalmic hydrogel preparation included the following steps:
(1) 50 mg of gelatin, 0.5 mg of a methylcobalamin TC, and 5 mg of proanthocyanidin were added to a centrifuge tube;
(2) 1 mL of water for injection was added to the centrifuge tube in step (1);
(3) a mixed system was dissolved completely by ultrasonication for 20 min in a water bath at 30° C. to 50° C. to form a uniform solution;
(4) a catalyst of 4.5 mg of carboxymethyl cellulose and 1.8 mg of an N-hydroxysulfosuccinimide sodium salt was added to the centrifuge tube in step (3); and
(5) the centrifuge tube containing the mixed solution obtained in step (4) was placed at a room temperature for 1 h to obtain the methylcobalamin ophthalmic hydrogel preparation.

### Example 3

A methylcobalamin ophthalmic liposome preparation included the following components:
a methylcobalamin TC 0.05%;
proanthocyanidin 0.1%;
lecithin 0.1%;
cholesterol 0.03%; and
pure water as a balance.

A preparation method of the methylcobalamin ophthalmic liposome preparation included the following steps:
(1) 15 mg of lecithin, 5 mg of cholesterol, 7.5 mg of methylcobalamin TC, and 1 mg of proanthocyanidin were added to the centrifuge tube;
(2) 10 mL of a mixture of methanol and chloroform (methanol:chloroform=1:3) was added to the centrifuge tube in step (1);
(3) a mixed system was completely dissolved by ultrasonication at a room temperature for 10 min to form a homogeneous solution, which was placed in a 10 mL round bottom flask; the homogeneous solution was distilled under reduced pressure at 100 rpm in a constant-temperature water bath at 30° C. to remove the oil solvent to form a thin film;
(4) the round-bottom flask in (3) was placed in a vacuum dryer overnight, added with 10 mL of a PBS (pH=7.4) the next day, and hydrated in a 37° C. water bath for 2 h to remove membrane of the liposomes;
(5) ultrasonication was conducted with a probe for 2 sec in an ice bath, with an interval of 3 sec, and the operation was repeated for 15 min to 20 min at a power of 200 W; and
(6) a product was centrifuged at 16,000 rpm for 40 min, a supernatant was discarded, and a precipitate was collected. After adding 15 ml of pure water, the precipitate was sonicated for 30 min, and passed through a microporous membrane with a pore size of 0.22 μm to obtain the methylcobalamin ophthalmic liposome preparation.

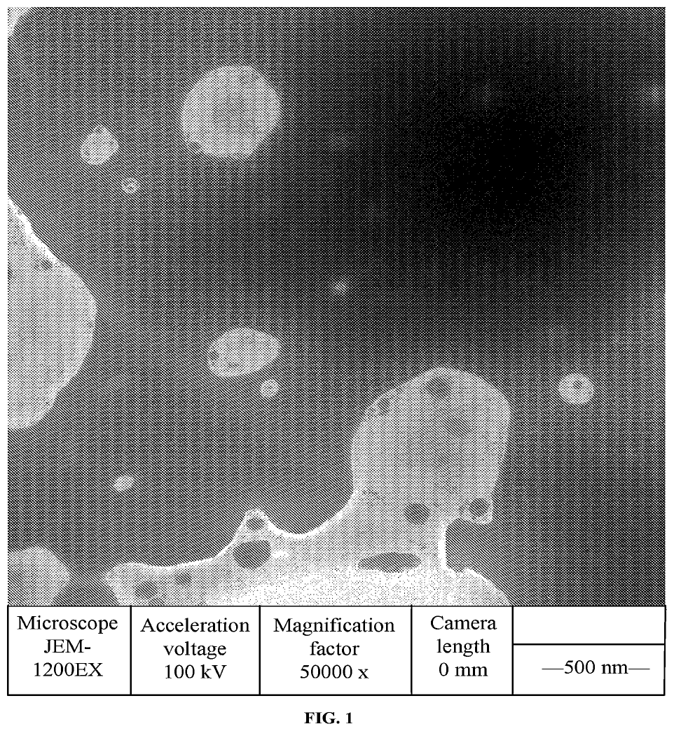

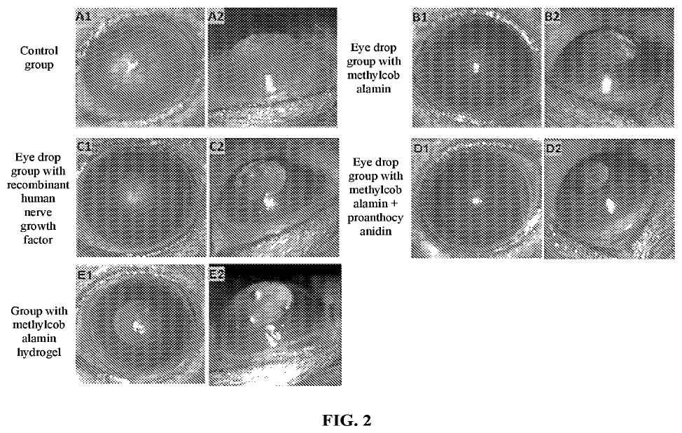

An electron micrograph of the methylcobalamin ophthalmic liposome preparation was shown in FIG. 1.

### Comparative Example 1

A control methylcobalamin ophthalmic drop included the following components:
a methylcobalamin TC 0.05%;
a buffer 0.1%;
an osmotic pressure regulator 0.5%;
a pH regulator 0.002%; and
water for injection as a balance; where
the methylcobalamin ophthalmic preparation had a pH value of 6.0 to 8.0.

A preparation method of the control methylcobalamin ophthalmic drop was the same as that in Example 1.

Performance Testing

The animal experiments of methylcobalamin ophthalmic preparation were as follows:

(1) C57BL/6 mice, 6 to 8 weeks old, male, weighing 20 g to 25 g, were purchased from Beijing Vital River Laboratory Animal Technology Co., Ltd. Animal breeding was conducted in the Animal Laboratory of the Eye Institute of Shandong First Medical University. The temperature was controlled at 22±2° C., the humidity was controlled at 60±10%, a light and dark cycle was 12 h, and food and drink were given ad libitum.

(2) Experimental grouping: 30 of 6- to 8-week-old C57BL/6 male mice (body weight 20 g to 25 g) were randomly divided into 5 groups, 6 in each group, respectively:
1. A control group
2. An eye drop group with the control methylcobalamin ophthalmic drop Comparative Example 1
3. An eye drop group with a recombinant human nerve growth factor
4. An eye drop group with methylcobalamin+proanthocyanidin (Example 1)
5. A subconjunctival implantation group with a methylcobalamin hydrogel (Example 2)

(3) Mouse Model of Neurotrophic Keratitis

The mice were intraperitoneally anesthetized with a 0.6% pentobarbital sodium solution (1 ml/100 g), fixed on an operating table in a lateral position, and the bulbar conjunctiva was incised 90 degrees along the temporal corneoscleral limbus with Vannas scissors. The retroorbital venous plexus should not be damaged. After carefully separating the fascia, lateral rectus muscle, and connective tissue with toothless micro-tweezers, the nasal conjunctival fornix was gently pushed with the toothless tweezers, and the eyes were rotated about 120 degrees to the nasal side. The cornea should not be damaged to expose the optic nerve. The ciliary nerve branch of the trigeminal nerve was located close to the optic nerve. After rotating the eyeball, under direct observation, the ciliary nerve was injured at the posterior sclera close to the optic nerve with sharp forceps. During the whole process, other tissues should not be injured, and the bulbar conjunctiva should be reset. Ofloxacin eye ointment was applied after the operation.

(4) Model Processing

The control group received eye drops with PBS solution, 4 times a day; the eye drop group with the control methylcobalamin ophthalmic drop was instilled with methylcobalamin eye drops every day, 4 times a day; the eye drop group with a recombinant human nerve growth factor was administered daily with the recombinant human nerve growth factor, 4 times a day; the eye drop group with methylcobalamin+proanthocyanidin was administered 4 times a day; in the subconjunctival implantation group with a methylcobalamin hydrogel, about 0.02 mg of methylcobalamin-loaded hydrogel slow-release material was implanted into the subconjunctiva.

(5) Slit Lamp Photography

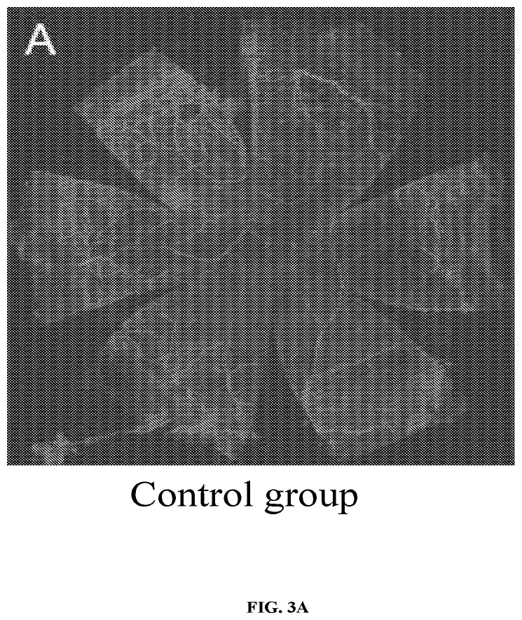

A representative general photo of the eye was taken on day 7 using a slit lamp for ophthalmic examination. FIG. 2 showed a general photo of the eyes of experimental mice. The results showed that in the control group, the corneal stroma was opaque, part of the corneal epithelium was defective, and neovascularization was more obvious. The corneal neovascularization of mice in the eye drop group with the control methylcobalamin ophthalmic drop, eye drop group with a recombinant human nerve growth factor, eye drop group with methylcobalamin+proanthocyanidin, and subconjunctival implantation group with a methylcobalamin hydrogel was not obvious. It was confirmed that the methylcobalamin ophthalmic preparation promoted the healing of the corneal epithelium and inhibited the formation of corneal neovascularization. It showed that in the eye drop group with methylcobalamin+proanthocyanidin, the area of corneal neovascularization in mice was reduced, the corneal epithelium healed, and the corneal transparency was better than that of the eye drop group with the control methylcobalamin ophthalmic drop, and a therapeutic effect was not weaker than that of the eye drop group with a recombinant human nerve growth factor. No obvious side effects were found during the treatment.

(6) Sampling

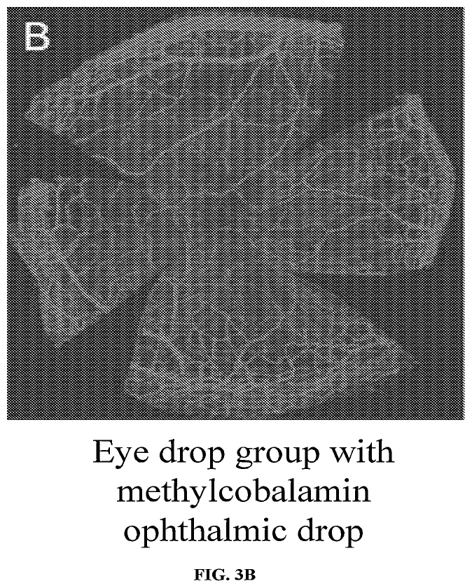

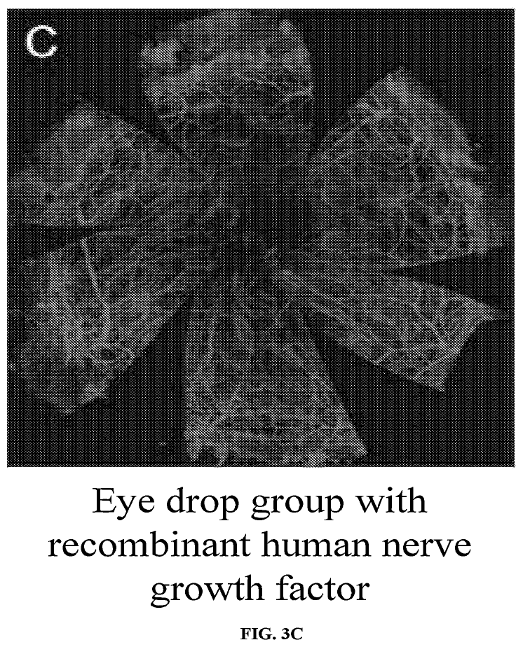

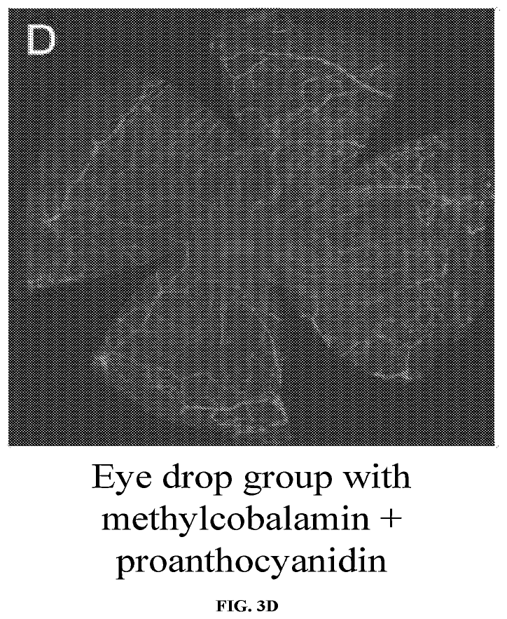

Samples were taken 14 d after the operation. The mice were sacrificed by decapitation, and the eyeballs of the mice were quickly and completely removed, and fixated in PBS containing 4% formaldehyde at 4° C. for 15 min. The excess tissues were trimmed and removed under a dissecting microscope to obtain corneal tissue with a complete horn-scleral limbus structure, and cut radially into 4 or 6 lobes to facilitate the flattening of corneal tissue, and fixated in the PBS containing 4% formaldehyde at 4° C. for 45 min. Corneas were washed 5 times in PBS, 10 min each time, and permeabilized and blocked with a blocking buffer (PBS containing 0.3% Triton-X-100) containing 20% goat serum for 1 h at a room temperature. Corneas were then incubated in blocking buffer of β-tubulin III for 2 h at a room temperature. The cornea was washed 6 times with PBS, 10 min each time, the corneal epithelium was spread upward, and sealed with DAPI-containing anti-fluorescence quenching buffered glycerin, stored in the dark, and then imaged with a confocal laser microscope. The results were shown in FIGS. 3A-D. The results showed that the corneal nerve endings in the control group did not regenerate, but the corneal nerve endings of the mice in the eye drop group with the control methylcobalamin ophthalmic drop, eye drop group with a recombinant human nerve growth factor, and eye drop group with methylcobalamin+proanthocyanidin all regenerated, confirming that the methylcobalamin ophthalmic preparation promoted the regeneration of corneal nerve endings. Moreover, in the eye drop group with methylcobalamin+proanthocyanidin, the density of regenerated nerves was better than that of the eye drop group with the control methylcobalamin ophthalmic drop, and the shape of regenerative nerves was better than that of the eye drop group with a recombinant human nerve growth factor.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A methylcobalamin ophthalmic preparation, comprising a methylcobalamin technical (TC) and proanthocyanidin, wherein the methylcobalamin TC and the proanthocyanidin have a mass ratio of (0.02-0.1):(0-0.5); and a dosage form of the methylcobalamin ophthalmic preparation is one or more of a drop, a hydrogel, or a liposome.

2. The methylcobalamin ophthalmic preparation according to claim 1, wherein when the dosage form is the drop, and the methylcobalamin ophthalmic preparation comprises the following components by percentage:
the methylcobalamin TC 0.02% to 0.1%;
the proanthocyanidin 0.1% to 0.5%;
a buffer 0.1% to 1.0%;
an osmotic pressure regulator 0.1% to 5.0%;
a pH regulator 0.001% to 0.004%; and
water for injection as a balance.

3. The methylcobalamin ophthalmic preparation according to claim 2, wherein the buffer is one or more selected from the group consisting of sodium hyaluronate, disodium hydrogen phosphate, sodium dihydrogen phosphate, potassium dihydrogen phosphate, and sodium citrate.

4. The methylcobalamin ophthalmic preparation according to claim 2, wherein the osmotic pressure regulator is one or more selected from the group consisting of propylene glycol, glycerin, and polyethylene glycol.

5. The methylcobalamin ophthalmic preparation according to claim 2, wherein the pH regulator is one or more selected from the group consisting of citric acid, sodium citrate, boric acid, disodium hydrogen phosphate, sodium dihydrogen phosphate, acetic acid, sodium acetate, sodium hydroxide, and hydrochloric acid.

6. The methylcobalamin ophthalmic preparation according to claim 1, wherein when the dosage form is the hydrogel, and the methylcobalamin ophthalmic preparation comprises the following components by percentage:
the methylcobalamin TC 0.02% to 0.1%;
the proanthocyanidin 0.1% to 0.5%;
gelatin 4.5% to 5.5%;
carboxymethyl cellulose 0.45% to 0.55%;
an N-hydroxysulfosuccinimide sodium salt 0.17% to 0.18%; and
water as a balance.

7. The methylcobalamin ophthalmic preparation according to claim 1, wherein when the dosage form is the liposome, and the methylcobalamin ophthalmic preparation comprises the following components by percentage:
the methylcobalamin TC 0.02% to 0.1%;
the proanthocyanidin 0.1% to 0.5%;
lecithin 0.08% to 0.15%;
cholesterol 0.02% to 0.05%; and
water as a balance.

8. The methylcobalamin ophthalmic preparation according to claim 1, wherein the methylcobalamin ophthalmic preparation has a pH value of 6 to 8.

9. The methylcobalamin ophthalmic preparation according to claim 1, wherein when the dosage form is the drop, and the methylcobalamin ophthalmic preparation has an osmolality of 260 mOsm/kg to 320 mOsm/kg.

10. A method for treating neurotrophic keratitis with a methylcobalamin ophthalmic preparation by using the methylcobalamin ophthalmic preparation according to claim 1.

11. The method according to claim 10, wherein when the dosage form is the drop, and the methylcobalamin ophthalmic preparation comprises the following components by percentage:
the methylcobalamin TC 0.02% to 0.1%;
the proanthocyanidin 0.1% to 0.5%;
a buffer 0.1% to 1.0%;
an osmotic pressure regulator
a pH regulator
water for injection

12. The method according to claim 11, wherein the buffer is one or more selected from the group consisting of sodium hyaluronate, disodium hydrogen phosphate, sodium dihydrogen phosphate, potassium dihydrogen phosphate, and sodium citrate.

13. The method according to claim 11, wherein the osmotic pressure regulator is one or more selected from the group consisting of propylene glycol, glycerin, and polyethylene glycol.

14. The method according to claim 11, wherein the pH regulator is one or more selected from the group consisting of citric acid, sodium citrate, boric acid, disodium hydrogen phosphate, sodium dihydrogen phosphate, acetic acid, sodium acetate, sodium hydroxide, and hydrochloric acid.

15. The method according to claim 10, wherein when the dosage form is the hydrogel, and the methylcobalamin ophthalmic preparation comprises the following components by percentage:
the methylcobalamin TC 0.02% to 0.1%;
the proanthocyanidin 0.1% to 0.5%;
gelatin 4.5% to 5.5%;
carboxymethyl cellulose 0.45% to 0.55%;
an N-hydroxysulfosuccinimide sodium salt 0.17% to 0.18%; and
water as a balance.

16. The method according to claim 10, wherein when the dosage form is the liposome, and the methylcobalamin ophthalmic preparation comprises the following components by percentage:
the methylcobalamin TC 0.02% to 0.1%;
the proanthocyanidin 0.1% to 0.5%;
lecithin 0.08% to 0.15%;
cholesterol 0.02% to 0.05%; and
water as a balance.

17. The method according to claim 10, wherein the methylcobalamin ophthalmic preparation has a pH value of 6 to 8.

18. The method according to claim 10, wherein when the dosage form is the drop, and the methylcobalamin ophthalmic preparation has an osmolality of 260 mOsm/kg to 320 mOsm/kg.
